(19) United States
(12) Patent Application Publication
(10) Pub. No.: US 2024/0390278 A1
(43) Pub. Date: Nov. 28, 2024

(54) PROCESS FOR MAKING A SOLVENT-FREE NANODISPERSION FOR FAT-SOLUBLE VITAMINS AND NUTRACEUTICALS

(71) Applicant: MALAYSIAN PALM OIL BOARD (MPOB), Kajang (MY)

(72) Inventors: Ju Yen FU, Kajang (MY); Puvaneswari MEGANATHAN, Kajang (MY); Kanga Rani SELVADURAY, Kajang (MY); Sivaruby KANAGARATNAM, Kajang (MY); Muhammad Roddy bin RAMLI, Kajang (MY); Rosidah binti RADZIAN, Kajang (MY)

(73) Assignee: MALAYSIAN PALM OIL BOARD (MPOB), Kajang (MY)

(21) Appl. No.: 18/201,107

(22) Filed: May 23, 2023

(30) Foreign Application Priority Data
Mar. 25, 2022 (MY) PI 2022001644

(51) Int. Cl. A61K 9/10; A23L 33/115; A23L 33/155; A61K 45/06

(52) U.S. Cl. CPC A61K 9/10; A23L 33/115; A23L 33/155; A61K 45/06

(57) ABSTRACT

The present invention relates to A solvent-free process for making a nanodispersion delivery system comprising emulsifying mixing fat soluble vitamins or nutraceuticals with miscible food grade lipids at a temperature of between 60-100° C. to obtain a lipid mixture, homogenizing said lipid mixture with a mixture of surfactants to form a coarse emulsion, downsizing said coarse emulsion to nanodispersion using ultrasonication or high pressure homogenization, and mixing said nanodispersion with shell/coating materials and spray drying.

### FIELD OF INVENTION

The present invention relates to processes for making nanodispersion delivery system.

### BACKGROUND OF THE INVENTION

Fat-soluble compounds such as vitamins and nutraceuticals having a continuous demand in the market for providing nutrition does not only demand providing and delivering large or sufficient amount of nutrients but also demands enhanced absorption in the body. Fat-soluble compounds upon oral consumption form unstable lipid aggregates which require bile salts for breaking said aggregates into micelles to be readily absorbed by the body. Hence, manufacturers of fat-soluble compounds have employed delivery vehicles such as nanodispersions in order to enable efficient delivery and absorption of fat-soluble compounds in the body.

US Patent Application US20130196852 uses a process for making a nanodispersion using an active agent, a carrier material, a stabilizing agent, a first solvent for the active agent and the stabilizing agent, and a second solvent for the carrier material. Said US Patent Application has a selection of carrier materials among inorganic materials, surfactants, polymers, and sugars, or mixtures of such. The process of said US application produces nano-coparticales of less than 320 nm and typically less than 250 nm having a polydispersity index (PDI) of 0.496.

United States Patent Application US20110217381 provides a solvent-free nanodispersion of an active in a carrier where such carrier has an enteric carrier soluble at intestinal pH but insoluble at stomach pH. Said nanodispersion is produced by mixing an active, a water-soluble carrier, said enteric carrier, a solvent for each active, and drying the mixture of said previous components.

US Patent Application US20200009067A1 provides a formulation and method for increasing the oral bioavailability of drugs by utilizing advanced pro-nano lipospheres (PNL) and PNL incorporating piperine. Said PNL containing drugs when taken orally and reaches the aqueous phase of the gastrointestinal (GI) tract, releases and spontaneously forms drug encapsulated nanoparticles with a particle diameter of 500 nm or less where such drug is a lipophilic compound or a mixture of two or more different lipophilic compounds.

Patent Application WO2012161562A1 relates to a process for producing nano-capsules and beadlets containing phytonutrients derived from palm oil. Such process comprises dissolving said phytonutrients in a non-polar solvent, adding at least one shell material and at least one additive into the solution, obtaining oil-in-water emulsion, removing the solvent from the mixture, homogenization and drying said mixture.

The abovementioned existing employing nanodispersions, when used to encapsulate fat-soluble vitamins/nutraceuticals allow the formation of stable particles without bile salts, enabling efficient absorption with minimum dependence on endogenous variability. Besides, dispersions in nanosize increased the surface area of vitamins/nutraceuticals by manifold. Thus, enhanced interaction with tissues and cells can be achieved at lower dose compared to the bulk material. However, the production of such nanodispersions often require solvent-based processes which require suitable environment for solvent handling and removal in the manufacturing facility which both solvent use and solvent handling has their costs that contribute to the production time and economics of nanodispersion based products or delivery systems. Furthermore, existing processes for making nanodispersions of fat-soluble compounds in water based products are highly dependent on the use of emulsifiers or emulsifying compounds.

While the abovementioned inventions or processes provide nanodispersions or nanodispersion delivery systems, such processes can further be streamlined in order to cut down material or reagent requirements, process steps, which when manufacturing rates and costs are being considered, will provide a much more efficient production of nanodispersion delivery systems for fat soluble compounds. The objective of the present invention is to provide a solvent-free process for making a nanodispersion delivery system.

### SUMMARY OF THE INVENTION

In one aspect, the invention provides a solvent-free process for making a nanodispersion delivery system comprising mixing fat soluble vitamins or nutraceuticals with miscible food grade lipids at a temperature of between 60-100° C. to obtain a lipid mixture, homogenizing said lipid mixture with a mixture of surfactants to form a coarse emulsion, downsizing said coarse emulsion to nanodispersion using ultrasonication or high pressure homogenization, and mixing said nanodispersion with shell/coating materials and spray drying.

In an aspect of the invention, said nanodispersion obtained from down-sizing in said solvent-free process for making a nanodispersion delivery system comprises a particle size of 50 to 1000nm and are relatively stable at zeta potential>|30|mV, and also has a high uniformity or having a PDI less than 0.4.

In an aspect of the invention, said miscible food grade lipids are selected from the group consisting of amphipathic lipids with hydrophilic portion and at least one lipophilic portion.

In an aspect of the invention, said surfactants are selected from the group consisting of long chain non-ionic surfactants with hydrophilic-lipophilic balance (HLB)>9.

In an aspect of the invention, said shell/coating materials are selected from the group consisting of carbohydrates and proteins.

In an aspect of the invention, said nanodispersion is for use in encapsulating fat-soluble compounds, or for encapsulating fat-soluble compounds selected from the group consisting of vitamins, nutraceuticals, carotenoids, co-enzyme Q10, and fish oils

### DETAILED DESCRIPTION OF THE INVENTION

Further understanding of the object, construction, characteristics and functions of the invention, a detailed description with reference to the embodiments is given in the following.

The present invention provides a solvent-free process for making a nanodispersion delivery system. Said process comprises mixing fat soluble vitamins or nutraceuticals with miscible food grade lipids at a temperature of between 60-100° C. to obtain a lipid mixture, homogenizing said lipid mixture with a mixture of surfactants to form a coarse emulsion, downsizing said coarse emulsion to nanodispersion using ultrasonication or high pressure homogenization, and mixing said nanodispersion with shell/coating materials and spray drying.

In one embodiment, said food grade lipids used in the present invention are selected from the group consisting of amphipathic lipids with hydrophilic portion and at least one lipophilic portion. In a preferred embodiment, said food grade lipids or amphipathic lipids with hydrophilic portion and at least one lipophilic portion are fatty esters, acetyl esters, monoglycerides of fatty acids, diglycerides of fatty acids, polyglycerol esters of fatty acids, phospholipids, sphingolipids, and lecithin, wherein the most preferred are monoglycerides of fatty acids. Monoglycerides are a form food emulsifiers produced using natural edible oils and fats. Being single chain lipids, the advantages of monoglycerides are narrow melting points suitable for oral application and high stability.

In one embodiment, said surfactants used in the present invention are wherein said surfactants are long chain non-ionic surfactants with hydrophilic-lipophilic balance (HLB)>9 selected from the group consisting of polyoxylglycerides, sorbitan esters, polyoxyethylene sorbitan esters, polyethylene glycol esters, polyglycerol esters, polyoxypropylene and polyoxyethylene copolymers, and sucrose esters.

In one embodiment, said shell/coating materials are carbohydrates. Said carbohydrates are selected from the group consisting of maltodextrin, modified starch, whey protein, xanthan gum, acacia gum, pectin, casein, wherein the most preferred are maltodextrin and sodium casein. Maltodextrin is able to withstand higher spray drying temperature compared to other carbohydrates such as sucrose or glucose, leading to higher yield and less caking problem in the spray-dried powder. Maltodextrin also improves the water solubility of spray-dried powder. Proteins have film-forming properties able to reduce particle-to-wall stickiness during spray drying. Casein produced spray-dried products with higher stability and yield.

Emulsifying or obtaining a first coarse emulsion from homogenized lipid mixture and surfactants were performed by low shear mixing between 1000-3000 rpm.

Down-sizing of said coarse emulsion into nanodispersion is performed using ultrasonication or high pressure homogenization. Nanodispersions producing at this step has a measured particle size ranging from 50 to 1000 nm and are relatively stable at zeta potential>|30|mV. As the nanodispersion produced displays high uniformity (PDI<0.4), a purification step is not anymore necessary unlike existing nanodispersion making methods which require both equipment and reactants for purification step. Down-sizing by ultrasonication is performed using a probe sonicator with energy input between 100 to 400 Watt second/gram (Ws/g). Down-sizing by high pressure homogenization is performed using pressure between 10,000 to 25,000 psi for 3 to 5 passes. As polydispersity index (PDI) indicates the heterogeneity of the distribution of nanoparticle size from 0-1, lower PDI indicates better homogeneity of nanoparticle sizes typically<0.4. Current prior art inventions provide nanodisperions and methods for producing nanodispersions of more than 0.4 to which the current invention has advantage over.

Coating said nanodispersion with shell materials or coating materials is performed using high shear mixing between 5000 to 15,000 rpm. Shell materials or coating materials which are adapted to be used with the invention is selected from the group consisting of food grade carbohydrates and proteins.

Spray drying said nanodispersion is further performed in order to further preserve the stability of the nanodispersion while maintaining the particle size at 50-1000 nm even 15 after reconstitution of nanodispersion powder in aqueous medium. After coating step, the coated or shelled nanodispersion is spray-dried using inlet temperature ranging between 80 to 200° C. at a feed flow rate between 5-25 mL/min depending on pump settings. Spray drying allows storage of said nanodispersion at ambient temperature (up to 25 ⋅C) for up to 3 months.

In a preferred embodiment, said solvent-free process for making a nanodispersion delivery system of the invention yields a nanodispersion powder of more than 60% after spray drying with reasonably high powder flowability and moisture content of less than 5%. Yield (%) is calculated as weight of powder collected from spray dryer (g)/weight of 25 total solid ingredients (g)×100%. Powder flowability was measured using Hausner ratio less than 1.25 and Carr's index less than 20%.

In one embodiment, the invention further comprises a nanodispersion obtainable by the solvent-free process for making a nanodispersion delivery system of the invention is provided. Said nanodispersion has a particle size of 50-1000 nm and a zeta potential of>|30| mV and also said nanoparticles of the nanodispersion has a high uniformity (polydispersity index of less than 0.4).

In one embodiment, said nanodispersion of the present invention, or nanodispersion obtained from the solvent-free process for making a nanodispersion delivery system of the invention is suitable to be used in encapsulating fat-soluble compounds selected from the group consisting of vitamins, nutraceuticals, carotenoids, co-enzyme Q10, fish oils, edible oils, vegetable oils. With nanodispersions, fat-soluble vitamins/nutraceuticals form stable particles without bile salts, enabling efficient absorption with minimum dependence on endogenous variability. Besides, dispersions in nanosize increased the surface area of vitamins/nutraceuticals by manifold. Thus, enhanced interaction with tissues and cells can be achieved at lower dose compared to the bulk material.

In another embodiment said nanodispersion of the present invention, or nanodispersion obtained from the solvent-free process for making a nanodispersion delivery system of the invention is suitable to be used for nanodispersion based fat-soluble vitamins/nutraceuticals in water-based products. Formulation of fat-soluble compounds in water-based product is often challenging due to their immiscible characteristics, causing phase separation of products. Conventional method to overcome this issue is emulsification where high amount of excipients/emulsifiers are used to ensure stability of products. Said water-based products is selected from the group consisting of beverages, instant beverage mixtures, emulsion, cream, cereal, sachet supplements and other food products.

In one embodiment said nanodispersion of the present invention, or nanodispersion obtained from the solvent-free process for making a nanodispersion delivery system of the invention is suitable to be used for administration via parenteral route (intravenous/subcutaneous/intraperitoneal/intramuscular) which require particle size to be less than 10 μm. Fat-soluble vitamins often required emulsification step to achieve this, which is highly dependent on emulsifiers, concentration of excipients, and thermodynamic stability of end products. Nanodispersion is readily dispersed in water-based matrix of fat-soluble compounds and are minimally affected by formulation challenges due to their high stability.

### Working Examples

### Preparation of Vitamin E Tocotrienols Nanodispersion

In an example provided, said process of making a nanodispersion delivery system is performed by the following steps:
1. Melt 50 g of glycerol monostearate at 70° C.
2. Warm 250 g of vitamin E tocotrienols at 70° C.
3. Warm Poloxamer 188 solutions ranging from 2.5 to 10% w/v at 70° C.
4. Emulsify glycerol monostearate, vitamin E tocotrienols and 700 mL of Poloxamer 188 solutions using low shear mixing at 1000 rpm, 70° C. for 15 minutes or until homogenous.
5. Probe sonicate the mixture using energy input of 200 watt second per gram (Ws/g) using Hielscher ultrasonicator.
6. Cool the mixture to room temperature.
7. To 100 mL of the mixture, add 20 g maltodextrin, 5 g of sodium casein and 50 ml of distilled water.
8. Homogenize the mixture at 5000 rpm for 20 minutes.
9. Spray dry at inlet temperature of 80° C., outlet temperature of 73° C., feed flow rate of 50 mL/hour, using Buchii B-290 spray dryer.

### Tests Conducted

### Particle Size

Particle size of nanodispersions were NanoZS Zetasizer (Malvern Instruments, Malvern, UK) after dilution×10000 in distilled water. Water for dilution was pre-filtered with 0.45 μm prior to analysis. All measurements were taken at 25° C. Spray-dried nanodispersion powder was prepared at 10 mg/mL prior to analysis.

| | Surfactant | Average | Polydispersity |
|---|---|---|---|
| | concentration | diameter | Index |
| No | (% w/v) | (nm) | (PDI) |
| 1 | 1.25 | 369.8 ± 10.6 | 0.444 ± 0.04 |
| 2 | 2.5 | 261.9 ± 4.9 | 0.335 ± 0.01 |
| 3 | 4 | 236.4 ± 1.9 | 0.279 ± 0.02 |
| 4 | 5 | 190.0 ± 0.8 | 0.210 ± 0.01 |
| 5 | 7.5 | 169.0 ± 4.8 | 0.197 ± 0.02 |

### Stability Test

Nanodispersion were stored at room temperature (25-28° C.) for 3 months. Particle size were measured according to methods described above.

| | | Average diameter |
|---|---|---|
| No | Month | (nm) |
| 1 | 0 | 143 |
| 2 | 3 | 148 |

### Powder Characteristics

Powder flowability was determined based on tapped density and bulk density measured according to USP method. Tapped density was found to be 400 kg/m³ while bulk density was found to be 500 kg/m³. Hausner ratio was measured at 1.25 using equation Tapped density/Bulk density. Carr's index was measured at 20% using equation (Tapped density−Bulk Density)/Tapped density×100%. Both indicators showed good powder flowability. Yield was calculated to be 76.3% using equation weight of powder collected from spray dryer (g)/weight of total solid ingredients (g)×100%. Moisture content was measured using standard method (AOCS Method Ca 2C-25) and found to be 1.01%.

As would be apparent to a person having ordinary skilled in the art, the afore-described composition may be provided in many variations, modifications or alternatives relating to solvent-free process for making a nanodispersion delivery system. The principles and concepts disclosed herein may also be implemented in various manner which may not have been specifically described herein but which are to be understood as encompassed within the scope and letter of the following claims.

1. A solvent-free process for making a nanodispersion delivery system comprising the steps,
mixing fat soluble vitamins or nutraceuticals with miscible food grade lipids at a temperature of between 60-100° C. to obtain a lipid mixture;
homogenizing said lipid mixture with a mixture of surfactants to form a coarse emulsion;
downsizing said coarse emulsion to nanodispersion using ultrasonication or high pressure homogenization; and
mixing said nanodispersion with shell/coating materials and spray drying.

2. The solvent-free process for making a nanodispersion delivery system of claim 1, wherein said nanodispersion obtained from down-sizing comprises a particle size of 50 to 1000 nm and are relatively stable at zeta potential>|30|mV.

3. The solvent-free process for making a nanodispersion delivery system of claim 1, wherein said nanodispersion obtained from down-sizing comprises high uniformity or having a PDI less than 0.4.

4. The solvent-free process for making a nanodispersion delivery system of claim 1, wherein said miscible food grade lipids are amphipathic lipids with hydrophilic portion and at least one lipophilic portion selected from the group consisting of fatty esters, acetyl esters, monoglycerides of fatty acids, diglycerides of fatty acids, polyglycerol esters of fatty acids, phospholipids, sphingolipids, and lecithin, wherein the most preferred are monoglycerides of fatty acids.

5. The solvent-free process for making a nanodispersion delivery system of claim 1, wherein said surfactants are long chain non-ionic surfactants with HLB>9 selected from the group consisting of polyoxylglycerides, sorbitan esters, polyoxyethylene sorbitan esters, polyethylene glycol esters, polyglycerol esters, polyoxypropylene and polyoxyethylene copolymers, and sucrose esters.

6. The solvent-free process for making a nanodispersion delivery system of claim 1, wherein said shell/coating materials are selected from the group consisting of carbohydrates including maltodextrin, modified starch, whey protein, xanthan gum, acacia gum, pectin, and casein.

7. The solvent-free process for making a nanodispersion delivery system of claim 1, for use in encapsulating fat-soluble compounds.

8. The solvent-free process for making a nanodispersion delivery system of claim 7, wherein said fat-soluble compounds is selected from the group consisting of vitamins, nutraceuticals, carotenoids, co-enzyme Q10, fish oils, edible oils, and vegetable oils.
